(19) United States
(12) Patent Application Publication
(10) Pub. No.: US 2016/0324421 A1
(43) Pub. Date: Nov. 10, 2016

(54) Wearable LED Sensor Device that Employs a Matched Filter to Generate Biometric Samples

(71) Applicants: Ehsan Azarnasab, Palo Alto, CA (US); Abraham Carter, Palo Alto, CA (US); David Scott, North Salt Lake City, UT (US)

(72) Inventors: Ehsan Azarnasab, Palo Alto, CA (US); Abraham Carter, Palo Alto, CA (US); David Scott, North Salt Lake City, UT (US)

(21) Appl. No.: 15/066,134

(22) Filed: Mar. 10, 2016

Related U.S. Application Data
(60) Provisional application No. 62/157,810, filed on May 6, 2015.

(51) Int. Cl. A61B 5/00; G01J 1/44

(52) U.S. Cl. CPC A61B 5/0059; G01J 1/44; A61B 5/6824; A61B 5/7246; A61B 5/7278; A61B 2562/0238

(57) ABSTRACT

An LED of a wearable sensor device can be driven less frequently while still obtaining an accurate biometric sample. By reducing how frequently the LED is driven, the power required to operate the wearable sensor device is likewise reduced. To allow the LED to be driven less frequently while still obtaining an accurate biometric sample, a known lighting profile of an LED can be employed with a matched filter to generate a biometric sample from a number of samples taken while the LED is lighting. In this way, the LED only needs to be lighted for a short period of time and a reduced amount of data is stored both of which minimize the power requirements of the wearable sensor device.

### CROSS-REFERENCE TO RELATED APPLICATIONS

This application claims priority to U.S. Provisional Patent Application No. 62/157,810 which was filed on May 6, 2015.

### BACKGROUND

A wearable sensor device is a device worn by a user that is configured to monitor an action or characteristic of the user. For example, a wearable sensor device may include an accelerometer for detecting a user's movement and/or a biometric sensor for measuring the user's blood oxygen level or pulse rate. As wearable sensor devices become more commonplace, a primary design consideration is the power requirement of the wearable sensor device. If a wearable sensor device does not provide good battery life, it can be inadequate for monitoring a user's biometric characteristics. Accordingly, the present invention is directed to techniques for increasing the power efficiency of a wearable sensor device, and in particular, of a wearable sensor device that employs LEDs and a light sensor to detect a wearer's biometrics.

### BRIEF SUMMARY

The present invention extends to wearable sensor devices and to methods performed by such wearable sensor devices which increase the power efficiency of the wearable sensor device. The present invention includes techniques for allowing an LED of a wearable sensor device to be driven less frequently while still obtaining an accurate biometric sample. By reducing how frequently the LED is driven, the power required to operate the wearable sensor device is likewise reduced.

To allow the LED to be driven less frequently while still obtaining an accurate biometric sample, the present invention can employ a known lighting profile of an LED in conjunction with a matched filter to generate a biometric sample from a number of samples taken while the LED is lighting. In this way, the LED only needs to be lighted for a short period of time and a reduced amount of data is stored both of which minimize the power requirements of the wearable sensor device.

In one embodiment, the present invention is implemented as a a wearable sensor device that includes a housing configured to allow the wearable sensor device to be worn on a portion of the body, and a circuit for producing biometric samples. The circuit includes a first LED secured to the housing in a manner that causes the first LED to face the portion of the body when the wearable sensor device is worn; a light sensor secured to the housing, the light sensor being positioned to receive light that is transmitted from the first LED and reflected from the portion of the body, the light sensor being configured to generate a first set of sensed samples representing an intensity of the reflected light during a first period of time; a matched filter configured to receive the first set of sensed samples and a known lighting profile of the first LED, the matched filter performing a weighted average of the first set of sensed samples and the known lighting profile to generate a first biometric sample for the first period of time; and a storage for storing the first biometric sample.

In another embodiment, the present invention is implemented as a method for generating a biometric sample. A first LED is powered on at a first time. A first set of sensed samples is received from a light sensor. The first set of sensed samples represents an intensity of light from the first LED that is incident on the light sensor over a first period of time after the first time. A known lighting profile of the first LED is received. A biometric sample is generated from the first set of sensed samples and the known lighting profile of the first LED.

In another embodiment, the present invention is implemented as a wearable sensor device that includes a housing configured to allow the wearable sensor device to be worn on a portion of the body, and a circuit for producing biometric samples. The circuit includes a first LED and a second LED that are each secured to the housing in a manner that causes the first LED and the second LED to face the portion of the body when the wearable sensor device is worn; a light sensor secured to the housing, the light sensor being positioned to receive light that is transmitted from the first and second LEDs and reflected from the portion of the body, the light sensor being configured to generate, during a first period of time, a first set of sensed samples representing an intensity of the reflected light of the first LED and a second set of sensed samples representing an intensity of the reflected light of the second LED; and a matched filter configured to receive the first set of sensed samples and a known lighting profile of the first LED and to receive the second set of sensed samples and a known lighting profile of the second LED, the matched filter configured to generate a first biometric sample and a second biometric sample for the first period of time, the first biometric sample being generated by performing a weighted average of the first set of sensed samples and the known lighting profile of the first LED, the second biometric sample being generated by performing a weighted average of the second set of sensed samples and the known lighting profile of the second LED.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example of a bracelet that can be configured to implement embodiments of the present invention;

FIG. 2 illustrates an example circuit diagram for implementing embodiments of the present invention;

FIG. 3 illustrates lighting profiles for a red and infrared LED; and

FIG. 4 is a flowchart of an example method for generating a biometric sample.

### DETAILED DESCRIPTION

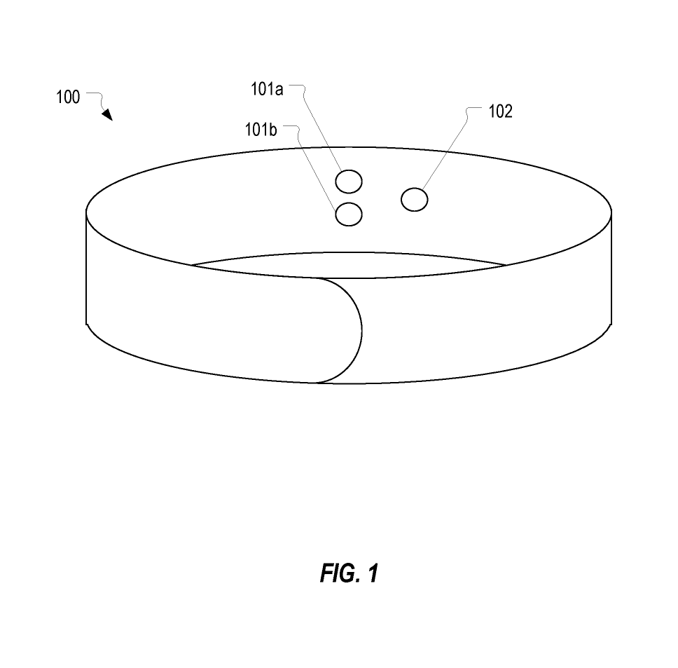

FIG. 1 illustrates an example of a bracelet 100 that can be configured to implement embodiments of the present invention. Although a bracelet configured to be worn around the wrist will be used to describe the present invention, it is noted that other types of wearable devices that can be worn on the wrist or other parts of the body can also be configured to perform embodiments of the present invention.

Bracelet 100 includes a red LED 101a and an infrared (IR) LED 101b that are exposed on an inner surface of bracelet 100. Accordingly, when bracelet 100 is worn by a user, red LED 101a and IR LED 101b will emit red light and infrared waves (collectively referred to as “light”) onto the wearer's skin.

Bracelet 100 also includes a light sensor 102 that is exposed on the inner surface of bracelet 100. Light sensor 102 is positioned adjacent LEDs 101a, 101b so as to be able to capture light (i.e., both red light and infrared waves) that is emitted by LEDs 101a, 101b and reflected from the wearer's body. By sensing light that is reflected from the wearer's body, one or more biometrics of the wearer can be determined. These biometrics can include, for example, heart rate, heart rate variability, respiratory rate, oxygen saturation, pulse pressure, systemic vascular resistance, arterial stiffness, stroke volume, systolic blood pressure, diastolic blood pressure, cardiac output, and left ventricular ejection fraction.

A primary design consideration for a wearable device is its battery life. In bracelet 100, the primary components that consume power are the LEDs. Accordingly, if the LEDs can be driven less frequently, the battery life of bracelet 100 will be increased. However, driving the LEDs less frequently would typically reduce the quality of the samples or require substantial and expensive circuitry to produce quality samples. The present invention is primary directed to these issues, or in other words, the present invention provides a way to drive the LEDs less frequently while still obtaining quality biometric samples.

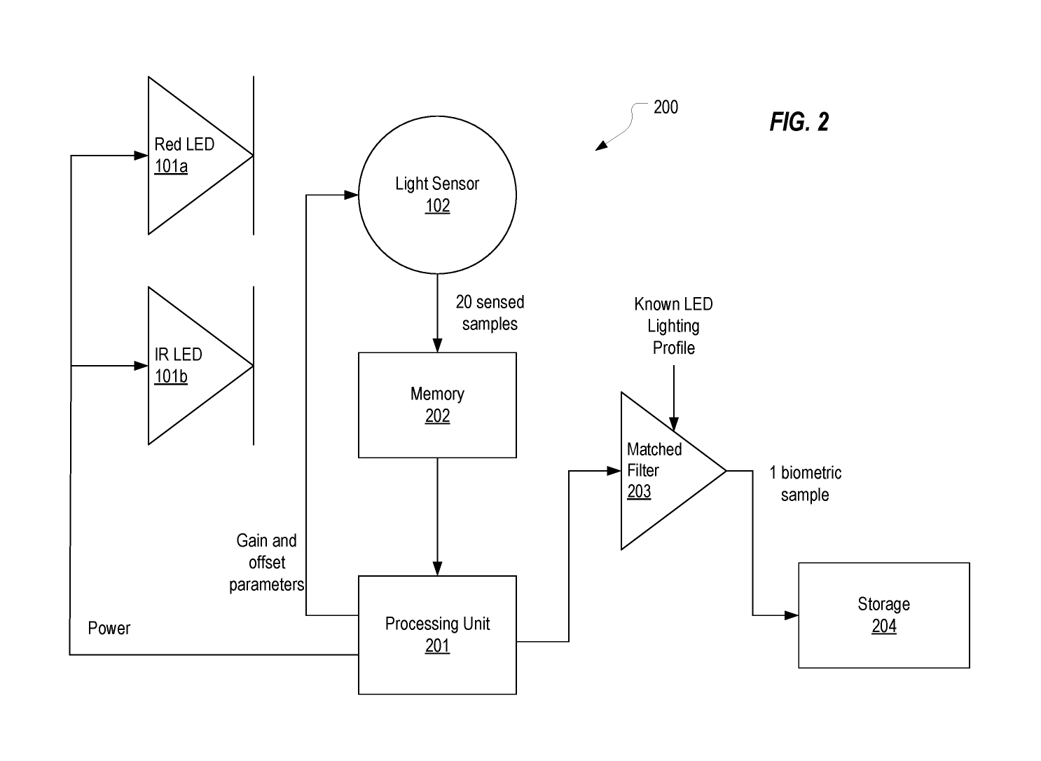

FIG. 2 illustrates a block diagram of a circuit 200 that can be used to generate biometric samples from LEDs 101a, 101b even when the LEDs are only powered on for short periodic durations. Circuit 200 includes red LED 101a, IR LED 101b, and light sensor 102 as depicted in FIG. 1. As described above, light sensor 102 is configured to sense light that is emitted from LEDs 101a, 101b and reflected from the wearer's body. Light sensor 102 provides an output (e.g., a voltage) that represents the intensity of light that is incident on the light sensor. Light sensor 102 can be configured to provide a first output representing the intensity of red light and a second output representing the intensity of the infrared waves. Readings of the outputs of light sensor 102 will be referred to herein as “sensed samples” and are to be distinguished from biometric samples which are generated from sensed samples as will be described below.

The remaining components of circuit 200 are configured to allow LEDs 101a, 101b to be driven at short periodic intervals while still generating useful biometric samples. For purposes of the current discussion, it will be assumed that circuit 200 is configured to generate a single biometric sample per LED every 33 ms (i.e., to produce biometric samples at a frequency of 30 Hz). However, biometric samples could equally be produced at different frequencies in accordance with embodiments of the present invention. Accordingly, for every cycle that LEDs 101a, 101b are powered on, a single biometric sample will be generated based on sensed samples of red LED 101a and a single biometric sample will be generated based on sensed samples of IR LED 101b.

Because biometric samples are produced at a frequency of 30 Hz, circuit 200 can include a processing unit 201 for driving LEDs 101a, 101b at a frequency of 30 Hz. Processing unit 201 can therefore power on LEDs 101a, 101b every 33 ms (e.g., by employing an interrupt service routine). At each cycle, processing unit 201 can drive LEDs 101a, 101b for a specified duration to allow a sufficient number of sensed samples to be obtained. This specified duration can correspond with the amount of time that it takes for LEDs 101a, 101b to fully power up (i.e., to reach full intensity after being in an off state). In some embodiments, this duration may be approximately 3 ms which is slightly longer than the amount of time it takes for LEDs 101a, 101b to reach full intensity (which is typically around 1.5 ms).

Circuit 200 can include memory 202 in which sensed samples are temporarily stored. In conjunction with driving LEDs 101a, 101b each cycle, processing unit 201 can also initiate direct memory access to cause a number of sensed samples to be written to memory 202 each cycle. For example, every 33 ms, direct memory access may be enabled to cause 20 sensed samples per LED to be stored in memory 202. Accordingly, a total of 600 sensed samples would be generated per LED per second. In a particular embodiment, the sampling interval employed to obtain sensed samples may be approximately 0.068 ms such that the 20 sensed samples are obtained over a period of approximately 1.36 ms.

As stated above, a single biometric sample per LED is generated for each cycle. Therefore, a biometric sample generated for a given cycle is based on the 20 corresponding sensed samples obtained during the given cycle. Although it may be possible to do a simple averaging of the 20 sensed samples or to take the highest value from the 20 sensed samples to generate the biometric sample, the present invention employs a matched filter to generate the biometric sample from the sensed samples as a weighted average as will now be described. By employing a matched filter to implement a weighted average, an accurate biometric sample can be generated even though the biometric sample is based on sensed samples obtained while the LED is powering on.

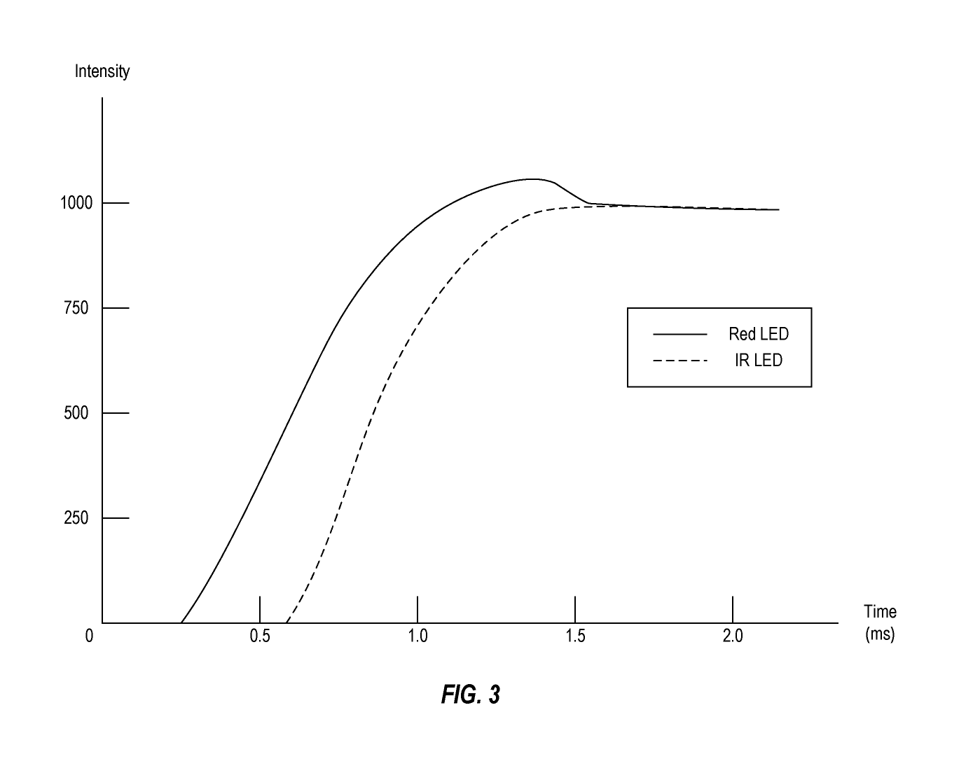

FIG. 3 illustrates example lighting profiles of red LED 101a and IR LED 101b. These lighting profiles represent how the intensity of the light emitted by the LEDs increases when the LEDs are powered on. In other words, these lighting profiles can represent the output of light sensor 102 that would be generated when light sensor 102 senses unobstructed light emitted from LEDs 101a, 101b while the LEDs are powered on. In the graph in FIG. 3, each LED is powered on at time 0 ms at which point the intensity of each LED is 0. Each LED has a delay before any light is emitted. For example, red LED 101a is shown as having a delay of approximately 0.2 ms while IR LED 101b is shown as having a delay of approximately 0.7 ms. After these delays, the intensity of the light emitted by each LED increases quickly until reaching a steady-state value of approximately 1000.

To employ these lighting profiles to generate biometric samples from the sensed samples, the sensed samples and the lighting profiles can be represented as vectors having the same dimensions. In the current example, each vector would therefore comprise 20 entries. For example, vectors representing the known lighting profile of red LED 101a (hereinafter “Red Template”) and the known lighting profile of IR LED 101b (hereinafter “IR Template”) can be as follows:

Red Template=[0,0,0,13,38,61,77,88,95,99,101,103,103,103,103,97,97,97,96,96]

IR Template=[0,0,0,0,0,0,11,33,59,74,83,89,92,94,95,96,96,96,96,96]

As shown, in some embodiments, the template values can be scaled down (e.g., by a factor of 10) to prevent overflow during the matched filter processing.

Circuit 200 can be configured to provide the red template and IR template as inputs to matched filter 203 as shown in FIG. 2. At each cycle, processing unit 201 can be configured to access memory 202 to obtain the 20 sensed samples per LED and provide the sensed samples in the form of a vector. For example, example vectors containing sensed samples for red LED 101a (hereinafter “Red Samples”) and sensed samples for IR LED 101b (hereinafter “IR Samples”) for a particular cycle can be as follows:

Red Samples=[0,0,0,0,0,1,75,308,542,718,836,911,955,978,990,994,989,618,323,171]

IR Samples=[0,0,0,0,0,0,0,0,22,278,601,774,864,929,951,958,960,577,411,253]

Matched filter 203 can be configured to first perform a dot product on the template and sample vectors. For example, matched filter 203 can produce the dot product of Red Template and Red Samples to generate a result corresponding to red LED 101a and can produce the dot product of IR Template and IR Samples to generate a result corresponding to IR LED 101b. Matched filter 203 can then divide the dot product by the number of sensed samples to generate the biometric sample as a weighted average. In some embodiments, the dot product can be divided by an integer multiple of the number of samples to reduce the size of the biometric sample. By reducing the size of the biometric samples (e.g., by three bits), less power can be required to transmit and store the biometric sample in storage 204.

Accordingly, matched filter 204 can be configured to perform the following calculation:

$$\frac{Template \cdot Samples}{X} = Biometric Sample$$

where X is an integer multiple of the number of samples such as 200. Using 200 as the value of X, the biometric sample that would be generated from Red Template and Red Samples would therefore be 4672.

By employing the known lighting profiles in this calculation, the dynamic range of the biometric samples is increased while minimizing any effect that noise in the sensed samples will have on the calculation. For example, if it is assumed that the sensed samples are each represented as 11 bit values, matched filter 203 can yield a 14 bit value for the biometric sample. Employing the templates in the dot product calculation ensures that this increase in 3 bits represents a fixed gain that minimizes the presence of noise in the reflected light.

In some embodiments, the leading zeros in the templates can also be used to identify when bracelet 100 is not being worn. If bracelet 100 is not being worn when an LED cycle is initiated, the initial sensed samples may have a non-zero value due to ambient light incident on light sensor 102. In such cases, circuit 200 can be configured to detect that bracelet 100 is not being worn and can enter into a power saving mode in which the LEDs are not cycled on.

In some embodiments, processing unit 201 can be configured to monitor the values of the sensed samples to determine if the gain and offset of light sensor 102 and/or the power level of LEDs 101a, 101b should be adjusted. To maximize power savings, processing unit 201 can be configured to monitor the values of the sensed samples to determine if the values are sufficiently high, and if so, can lower the amount of power that is output to drive one or both of the LEDs. If the values of the sensed samples become too low, processing unit 201 can then increase the power level thereby increasing the intensity of the light emitted by the LEDs. This process can be continually performed to ensure that the LEDs are driven with just enough power to produce suitable sensed samples.

If adjusting the power level of the LEDs does not produce satisfactory increases in the values of the sensed samples, the gain of light sensor 102 can be increased. Also, if the baseline of the sensed samples moves, the offset of light sensor 102 can be adjusted accordingly. This control loop can be implemented to ensure that the quality of the sensed samples is maintained while also minimizing the amount of power that circuit 200 consumes.

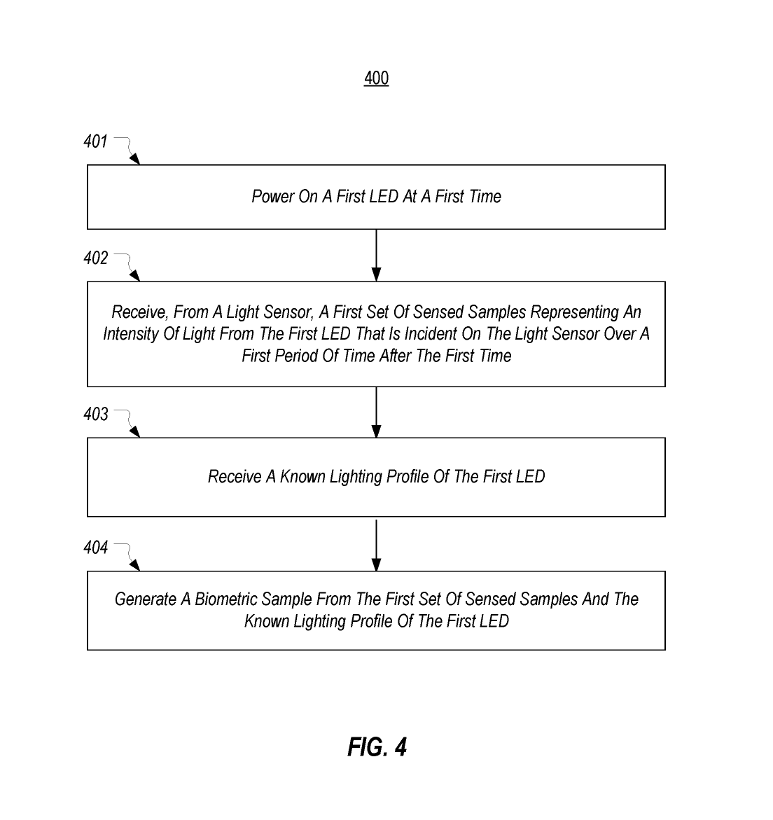

FIG. 4 provides a flowchart of an example method 400 generating a biometric sample. Method 400 will be described with reference to FIG. 2 and the example templates and sensed samples provided above.

Method 400 includes a step 401 of powering on a first LED at a first time. For example, red LED 101a or IR LED 101b could be powered on at a first time.

Method 400 includes an act of receiving, from a light sensor, a first set of sensed samples representing an intensity of light from the first LED that is incident on the light sensor over a first period of time after the first time. For example, matched filter 203 can receive the Red Samples vector or the IR Samples vector.

Method 400 includes an act 403 of receiving a known lighting profile of the first LED. For example, matched filter 203 can receive the Red Template vector or IR Template vector.

Method 400 includes an act 404 of generating a biometric sample from the first set of sensed samples and the known lighting profile of the first LED. For example, matched filter 203 can generate a biometric sample from Red Samples and Red Template or from IR Samples and IR Template.

The present invention has been described as employing a known lighting profile to generate a biometric sample. However, the same process for generating a biometric sample can be employed with a template that is not a known lighting profile. In other words, the values within the template do not need to correspond with the intensity of light emitted by the LED as it is powered on. For example, a generic template configured as a vector of twenty values could be employed in place of Red Template and/or IR Template in the above described example. For purposes of this description and the following claims, therefore, the term “template” should be construed as a vector of values whether or not the values represent a known lighting profile of an LED.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed:

1. A wearable sensor device comprising:
a housing configured to allow the wearable sensor device to be worn on a portion of the body; and
a circuit for producing biometric samples, the circuit comprising:
a first LED secured to the housing in a manner that causes the first LED to face the portion of the body when the wearable sensor device is worn;
a light sensor secured to the housing, the light sensor being positioned to receive light that is transmitted from the first LED and reflected from the portion of the body, the light sensor being configured to generate a first set of sensed samples representing an intensity of the reflected light during a first period of time;
a matched filter configured to receive the first set of sensed samples and a first template, the matched filter performing a weighted average of the first set of sensed samples and the template to generate a first biometric sample for the first period of time; and
a storage for storing the first biometric sample.

2. The wearable sensor device of claim 1, wherein the first template comprises a known lighting profile of the first LED.

3. The wearable sensor device of claim 1, wherein the circuit further comprises:
a second LED secured to the housing in a manner that causes the second LED to face the portion of the body when the wearable sensor device is worn;
the light sensor being configured to generate a second set of sensed samples representing an intensity of reflected light from the second LED during the first period of time; and
wherein the matched filter is further configured to receive the second set of sensed samples and a second template, the matched filter performing a weighted average of the second set of sensed samples and the second template to generate a second biometric sample for the first period of time.

4. The wearable sensor device of claim 3, wherein the second template comprises a known lighting profile of the second LED.

5. The wearable sensor device of claim 1, wherein the circuit further comprises: a processing unit that is configured to power on the first LED for the first period of time at a specified frequency.

6. The wearable sensor device of claim 5, wherein the circuit is configured to generate a first biometric sample each time the first LED is powered on.

7. The wearable sensor device of claim 5, wherein the first set of sensed samples are initially stored in memory using direct memory access and then provided to the matched filter.

8. The wearable sensor device of claim 1, wherein the first set of sensed samples and the first template each comprise a vector having the same size, and wherein performing a weighted average of the first set of sensed samples and the first template comprises generating a dot product of the first set of sensed samples and the first template and then dividing the result of the dot product by an integer multiple of the size.

9. The wearable sensor device of claim 2, wherein the known lighting profile of the first LED includes one or more zero values representing a delay in powering on the first LED.

10. The wearable sensor device of claim 1, wherein the first LED is one of a red LED or an IR LED.

11. The wearable sensor device of claim 1, wherein the circuit comprises a processing unit configured to adjust the gain or offset of the light sensor based on the sensed samples.

12. The wearable sensor device of claim 1, wherein the circuit comprises a processing unit configured to adjust a power level supplied to the first LED based on the sensed samples.

13. A method for generating a biometric sample comprising:
powering on a first LED at a first time;
receiving, from a light sensor, a first set of sensed samples representing an intensity of light from the first LED that is incident on the light sensor over a first period of time after the first time;
receiving a known lighting profile of the first LED; and
generating a biometric sample from the first set of sensed samples and the known lighting profile of the first LED.

14. The method of claim 13, wherein the biometric sample is generated by performing a weighted average of the first set of sensed samples and the known lighting profile.

15. The method of claim 14, wherein the weighted average is performed by calculating a dot product of the first set of sensed samples and the known lighting profile and then dividing the dot product by a multiple of the number of sensed samples in the first set.

16. The method of claim 13, wherein the known lighting profile comprises a number of values representing an increasing intensity of light that is emitted by the first LED as the first LED is powered on.

17. The method of claim 16, wherein the known lighting profile includes one or more zero values representing a delay when the first LED is powered on.

18. The method of claim 13, further comprising:
periodically powering on the first LED to periodically generate a corresponding first set of sensed samples; and
each time the first LED is powered on to generate a corresponding first set of sensed samples, generating a corresponding biometric sample.

19. The method of claim 13, wherein the first period of time is less than 3 ms.

20. The method of claim 13, wherein the first set of samples comprises at least 20 samples.

21. The method of claim 13, further comprising:
powering on a second LED at the first time;
receiving, from the light sensor, a second set of sensed samples representing an intensity of light from the second LED that is incident on the light sensor over the first period of time;
receiving a known lighting profile of the second LED; and
generating a second biometric sample from the second set of sensed samples and the known lighting profile of the second LED.

22. A wearable sensor device comprising:
a housing configured to allow the wearable sensor device to be worn on a portion of the body; and
a circuit for producing biometric samples, the circuit comprising:
a first LED and a second LED that are each secured to the housing in a manner that causes the first LED and the second LED to face the portion of the body when the wearable sensor device is worn;
a light sensor secured to the housing, the light sensor being positioned to receive light that is transmitted from the first and second LEDs and reflected from the portion of the body, the light sensor being configured to generate, during a first period of time, a first set of sensed samples representing an intensity of the reflected light of the first LED and a second set of sensed samples representing an intensity of the reflected light of the second LED; and
a matched filter configured to receive the first set of sensed samples and a first template corresponding to the first LED and to receive the second set of sensed samples and a second template corresponding to the second LED, the matched filter configured to generate a first biometric sample and a second biometric sample for the first period of time, the first biometric sample being generated by performing a weighted average of the first set of sensed samples and the first template, the second biometric sample being generated by performing a weighted average of the second set of sensed samples and the second template.

23. The wearable sensor device of claim 22, wherein the first template comprises a known lighting profile of the first LED and the second template comprises a known lighting profile of the second LED.
